(19) United States
(12) Patent Application Publication
(10) Pub. No.: US 2008/0306503 A1
(43) Pub. Date: Dec. 11, 2008

(54) MECHANICALLY DETACHABLE VASO-OCCLUSIVE DEVICE

(75) Inventors: Like Que, Livermore, CA (US); Esther Chang, Fremont, CA (US); Ann Huang, Fremont, CA (US); Thazin Win, Chicago, IL (US)

Correspondence Address: VISTA IP LAW GROUP LLP, 12930 Saratoga Avenue, Suite D-2, Saratoga, CA, 95070, US

(73) Assignee: Boston Scientific Scimed, Inc., Maple Grove, MN (US)

(21) Appl. No.: 11/942,913

(22) Filed: Nov. 20, 2007

Related U.S. Application Data
(60) Provisional application No. 60/866,592, filed on Nov. 20, 2006.

(51) Int. Cl. A61M 37/00

(52) U.S. Cl. 606/191

(57) ABSTRACT

A device for delivering an occlusive element such as a vaso-occlusive coil includes an elongate sheath. An elongate releasing member having a distal tip or end is moveable within a lumen of the elongate sheath. A filament is provided having first and second ends with each end being fixed relative to the elongate sheath to form a loop segment. The loop segment of the filament is passed through a securing member located on the proximal end of the occlusive element. The distal tip of the elongate releasing member is inserted into the portion of the loop segment that passes through the securing member to lock the occlusive element relative to the elongate sheath. The occlusive element is released by pulling the elongate releasing member proximally to retract the distal tip from the loop segment of the filament.

### RELATED APPLICATION DATA

The present application claims the benefit under 35 U.S.C. § 119 to U.S. provisional patent application Ser. No. 60/866,592, filed Nov. 20, 2006. The foregoing application is hereby incorporated by reference into the present application in its entirety.

### FIELD OF THE INVENTION

The field of the invention generally relates to systems and delivery devices for implanting vaso-occlusive devices for establishing an embolus or vascular occlusion in a vessel of a human or veterinary patient.

### BACKGROUND OF THE INVENTION

Vaso-occlusive devices or implants are used for a wide variety of reasons, including treatment of intra-vascular aneurysms. A common vaso-occlusive device takes the form of a soft, helically wound coil formed by winding a platinum (or platinum alloy) wire strand about a primary mandrel. The relative stiffness of the coil will depend, among other things, on its composition, the diameter of the wire strand, the diameter of the primary mandrel, and the pitch of the primary windings. The coil is then wrapped around a larger, secondary mandrel, and again heat treated to impart a secondary shape. For example, U.S. Pat. No. 4,994,069, issued to Ritchart et al., describes a vaso-occlusive coil that assumes a linear, helical primary shape when stretched for placement through the lumen of a delivery catheter, and a folded, convoluted secondary shape when released from the delivery catheter and deposited in the vasculature.

In order to deliver the vaso-occlusive coils to a desired site, e.g., an aneurysm, in the vasculature, it is well-known to first position a small profile, micro-catheter at the site using a steerable guidewire. Typically, the distal end of the micro-catheter is provided, either by the attending physician or by the manufacturer, with a selected pre-shaped bend, e.g., 45°, 90°, “J”, “S”, or other bending shape, depending on the particular anatomy of the patient, so that it will stay in a desired position for releasing one or more vaso-occlusive coil(s) into the aneurysm once the guidewire is withdrawn. A delivery or “pusher” wire is then passed through the micro-catheter, until a vaso-occlusive coil coupled to a distal end of the pusher wire is extended out of the distal end opening of the micro-catheter and into the aneurysm. The vaso-occlusive device is then released or “detached” from the end pusher wire, and the pusher wire is withdrawn back through the catheter. Depending on the particular needs of the patient, another occlusive device may then be pushed through the catheter and released at the same site.

One known way to release a vaso-occlusive coil from the end of the pusher wire is through the use of an electrolytically severable junction, which is a small exposed section or detachment zone located along a distal end portion of the pusher wire. The detachment zone is typically made of stainless steel and is located just proximal of the vaso-occlusive device. An electrolytically severable junction is susceptible to electrolysis and disintegrates when the pusher wire is electrically charged in the presence of an ionic solution, such as blood or other bodily fluids. Thus, once the detachment zone exits out of the catheter distal end and is exposed in the vessel blood pool of the patient, a current applied to the conductive pusher wire completes a circuit with an electrode attached to the patient's skin, or with a conductive needle inserted through the skin at a remote site, and the detachment zone disintegrates due to electrolysis.

U.S. Pat. No. 5,122,136 issued to Guglielmi, et al. discloses a device in which a portion of the guidewire connected between the tip and the body of the guidewire is comprised of stainless steel and exposed to the bloodstream so that upon continued application of a positive current to the exposed portion, the exposed portion is corroded away at least at one location and the tip is separated from the body of the guidewire. The guidewire and a microcatheter are thereafter removed leaving the guidewire tip embedded in the thrombus formed within the vascular cavity.

One perceived disadvantage with vaso-occlusive devices that are deployed using electrolytic detachment is that the electrolytic process requires a certain amount of time to elapse to effectuate release of the vaso-occlusive element. This time lag is also a perceived disadvantage for vaso-occlusive delivery devices that utilize thermal detachment mechanisms. U.S. Pat. No. 6,966,892 issued to Gandhi, et al. discloses a vaso-occlusive device that uses a thermal detachment system.

Another detachment modality used to deploy vaso-occlusive elements uses mechanical detachment. U.S. Pat. No. 5,800,453 issued to Gia discloses embolic coils that have a receiving slot on one end. A catheter control wire or pusher guidewire having a hook which engages the coil's receiving slot is used as a coil pusher to eject the coil at the chosen site. The coils may also be placed within the lumen with a catheter in a nose-to-tail fashion and pushed into the body lumen. Pushing the coil assembly via the pusher from the distal end of the catheter body uncouples the distal most coil.

Another example of a mechanical detachment system is disclosed in U.S. Pat. No. 5,800,455 issued to Palermo et al. Palermo et al. discloses a delivery system that includes a coil having a clasp located at one end. The clasp includes a passageway for a control wire. The clasp interlocks with another clasp located on a distal end of a pusher member. The control wire is withdrawn in the proximal direction to release the coil.

Still other mechanical detachments systems have been proposed that use a fiber segment that is pulled in the proximal direction to decoupled the fiber from the embolic coil device. Examples of these systems may be found in U.S. Patent Application Publication Nos. 2006/0025803 A1 (coiled fiber), 2006/0025802 A1 (U-shaped fiber), and 2006/0025801 A1 (detachment filament).

One problem with certain existing mechanical detachment systems is that the junction between the embolic element and the releasing member moves during the detachment process which may adversely impact the placement of the embolic element within the aneurysm. Another complication is that mechanical detachment systems tend to have a stiff main section that complicates accurate placement of the delivery system at the desired location, i.e., a stiff section of the pusher wire or the pusher wire/coil junction can cause a pre-shaped micro-catheter to kick back or recoil from the aneurysm. Mechanical detachment systems also are perceived by physicians as being harder to use than other devices. In addition, certain mechanical detachment systems may jeopardize the integrity of the embolic element (e.g. coil) after detachment.

There thus is a need for a vaso-occlusive delivery system that utilizes mechanical detachment yet does not suffer from the aforementioned deficiencies. Such a system should be easy to use yet provide for consistent detachment of embolic elements in the desired location. Moreover, the delivery system should be able to release the embolic element without any micro-catheter kick back or recoil or other movement resulting from the detachment operation.

### SUMMARY

In one embodiment, a device for delivering an occlusive element includes an occlusive element such as, for example, a vaso-occlusive coil having a securing member positioned at a proximal end thereof. The device includes an elongate sheath having a distal end and a proximal end and a lumen extending between the distal and proximal ends. An elongate releasing member is disposed within the lumen of the elongate sheath, the elongate releasing member including a proximal end and a distal end, the elongate releasing member being moveable within the lumen of the elongate sheath. A filament is provided having first and second ends secured relative to the elongate sheath so as to form a loop segment. The delivery device includes a locked state in which the occlusive element is fixed to the distal end of the elongate sheath and an unlocked state in which the occlusive element is free from the elongate sheath. The device is in a locked state when the loop segment passes through the securing member and the distal end of the elongate releasing member engages with the portion of the loop segment that passes through the securing member. The device is in an unlocked state when the distal end of the elongate releasing member is retracted proximally from the loop segment.

In another embodiment, a device for delivering an occlusive element includes an occlusive element such as a vaso-occlusive coil that has a securing member positioned at a proximal end thereof. The delivery device further includes an elongate sheath having a distal end and a proximal end and a lumen extending between the two ends. An elongate releasing member is disposed within the lumen of the elongate sheath, the elongate releasing member including a proximal end and a distal tip, the elongate releasing member being moveable within the lumen of the elongate sheath. A blocking member is disposed in a distal end of the elongate sheath. The device includes a filament having first and second ends fixedly interposed between the blocking member and the elongate sheath so as to form a loop segment. The device includes a locked state and an unlocked state, the locked state being formed when the loop segment passes through the securing member and the distal end of the elongate releasing member engages with the loop segment passing through the securing member. The unlocked state is formed when the distal end of the elongate releasing member is retracted proximally from the loop segment.

In yet another embodiment, a method of loading a vaso-occlusive coil on a delivery device includes the steps of providing a vaso-occlusive coil having a securing member disposed at a proximal end thereof. An elongate sheath is provided having a distal end and a proximal end and a lumen extending between the two ends. A filament is provided having first and second ends secured to a distal portion of the elongate sheath to form a loop segment in the filament. An elongate releasing member is provided within the lumen of the elongate sheath, the elongate releasing member being moveable within the lumen of the elongate sheath. The loop segment of the filament is then inserted through the securing member of the vaso-occlusive coil. The distal end of the elongate releasing member is then inserted through the portion of the loop segment that was inserted through the securing member so as to affix the vaso-occlusive coil relative to the elongate sheath.

### BRIEF DESCRIPTION OF THE DRAWINGS

FIG. 1A is a cross-sectional view of a device for delivering an occlusive element according to one embodiment.

FIG. 1B is a cross-sectional view of the distal end of a delivery device according to another embodiment.

FIG. 2A is a cross-sectional view of a device for delivering an occlusive element according to another embodiment.

FIG. 2B is a cross-sectional view of the distal end of a delivery device according to another embodiment.

FIG. 3 is a cross-sectional view of a distal end of a device for delivering an occlusive element according to another embodiment.

FIG. 4A is a cross-sectional view of a device for delivering an occlusive element according to another embodiment.

FIG. 4B is a cross-sectional view of the proximal end of a delivery device according to another embodiment.

FIG. 5 is a cross-sectional view of a device for delivering an occlusive element according to another embodiment.

FIG. 6 is a cross-sectional view of a blood vessel with an aneurysm. A vaso-occlusive coil is shown inserted into the aneurysm.

### DETAILED DESCRIPTION OF THE ILLUSTRATED EMBODIMENTS

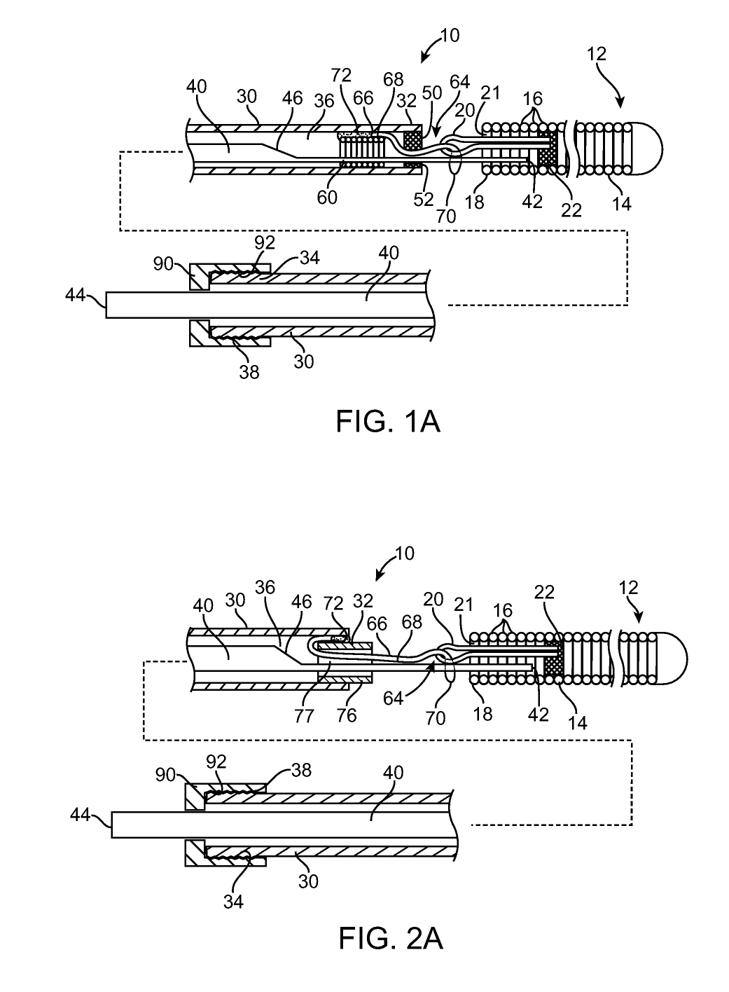

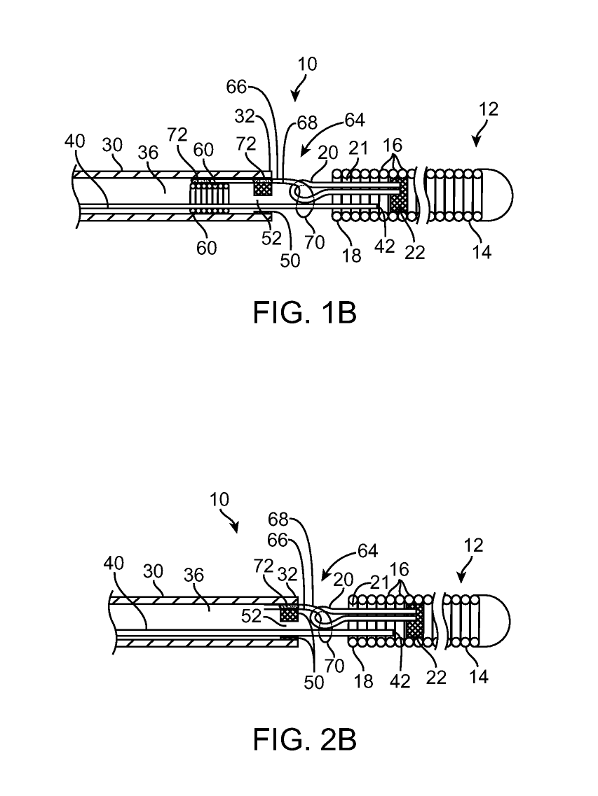

FIG. 1A illustrates a device 10 for delivering an occlusive element 12 to a vascular space such as, for example, aneurysm 100 (shown in FIG. 6). The occlusive element 12 may be formed as a vaso-occlusive coil 14 formed from a plurality of coil windings 16. When manufacturing the vaso-occlusive coil 14, the coil material is wound into a coil shape, which will typically be linear. Generally speaking, the coil 14 is a metallic coil made from a platinum alloy or a super-elastic alloy such as titanium/nickel alloy, known as “NITINOL.” The diameter of the wire used in the production of the coils 14 may fall in the range of about 0.00025 inches to about 0.006 inches. The coil 14 may have a primary diameter of between about 0.003 and about 0.025 inches, but for most neurovascular applications, a diameter between about 0.008 to about 0.018 inches provides sufficient hoop strength to hold the coil 14 in place within the chosen body site, lumen, or cavity, without substantially distending the wall of the site and without moving from the site as a result of the repetitive fluid pulsing found in the vascular system.

The axial length of the coil wire will usually fall in the range of around 0.5 to around 100 cm, more usually around 2.0 to 40 cm. Of course, all of the dimensions provided above should be viewed only as guidelines, and the invention, in its broader aspects, should not be limited thereto. Dimensions that are suitable for use in occluding sites within the human body are included in the scope of this invention.

Depending on the desired therapeutic effect and the shape of the site to be treated, the coil 14 may later be treated or accessorized in numerous ways in order to enhance its therapeutic effect. The coil 14 may be made to form various secondary shapes, often through the use of heat treatment, that may be better suited to fill a particular treatment site, as disclosed in U.S. Pat. Nos. 5,853,418 and 6,280,457, the entireties of which are expressly incorporated herein by reference. Alternatively, the coil 14 may have little or no shape after introduction into the vascular space, as disclosed in U.S. Pat. No. 5,690,666, the entirety of which is expressly incorporated by reference herein. In addition, external materials may be added to the outside of the coil 14 in an effort to increase its thrombolytic properties. These alternative embodiments are disclosed in U.S. Pat. Nos. 5,226,911, 5,304,194, 5,549,624, 5,382,259, and 6,280,457, the entireties of which are expressly incorporated herein by reference.

Still referring to FIG. 1A, the proximal end 18 of the coil 14 includes a securing member 20. The securing member 20 may be formed as a closed hoop, ring, or eyelet as is illustrated in FIG. 1A. For instance the securing member 20 may be formed using 0.002 inch platinum wire that is formed into a loop having an internal diameter on the order of around 0.005 inches. Alternatively, the securing member 20 may be formed in an open configuration such as a hook or the like (not shown). The hoop, ring, or eyelet form of the securing member 20 as seen in FIG. 1A has its two ends fixedly secured to a proximal end 18 of the coil 14. In one embodiment, the securing member 20 may be formed integrally with the coil 14. In this regard, the securing member 20 may be formed from a proximal winding of the coil 14. For example, the proximal winding may be looped back upon itself and optionally bonded to one or more windings 16 to form the closed securing member 20. Alternatively, as is shown in FIG. 1A, the securing member 20 may be formed separately from the coil 14. For example, the securing member 20 may be formed from a thin metal wire filament such as platinum, NITINOL, titanium, stainless steel, and metallic alloys. Alternatively, the securing member 20 may be formed form a polymer-based material such as PTFE, polypropylene, PEEK, and the like. The separate securing member 20 may then be jointed to the proximal end 18 of the coil 14 either by tying or through a bonding operation. FIG. 1A illustrates a bonding material 22 that is used to affix the securing member 20 to the coil 14. For instance, the bonding material 22 may include an adhesive material, solder, or weld.

In certain embodiments, such as the embodiments illustrated in FIGS. 1A, 1B, 2A, 2B, 3, 4A, and 6 (hidden) a pocket or lumen 21 allow passage of an elongate releasing member (discussed in more detail below). In still another alternative aspect, a short ring or segment of tubing (e.g., hypotube) may be located in the proximal end 18 of the coil 14. The ring or tubing (not shown) provides an access passageway that permits the elongate releasing member to pass at least partially within the interior of the coil 14. By permitting the elongate releasing member to enter the coil 14 this serves as a safety feature to prevent the same from poking or puncturing the aneurysm 100 (as shown in FIG. 6).

The delivery device 10 also includes an elongate sheath 30 having a distal end 32, a proximal end 34, and a lumen 36 therebetween. The elongate sheath 30 may be formed from a flexible yet lubricious material such as polyimide, polytetrafluoroethylene (PTFE), polyetheretherketone (PEEK), fluorinated ethylene propylene (FEP), or the like. Alternatively, the elongate sheath 30 may be formed from non-polymer materials such as hypotube material (e.g., metallic hypotube such as stainless steel or NITINOL). In yet another alternative, the elongate sheath 30 may be made from a combination of metallic and polymer materials, i.e., a composite structure. For example, stainless steel hypotube having an internal diameter of around 0.010 inches and an outer diameter of around 0.13 inches may be used. The elongate sheath 30 generally has a length that permits the same to be advanced intravascularly to the site of interest. For example, the elongate sheath 30 has a length to permit the distal end 32 to be positioned adjacent to the delivery site (e.g., aneurysm 100) while the proximal end 34 is positioned outside the patient's body. A typical range of lengths for the elongate sheath 30 may include between about 1.25 meters to about 2.0 meters.

Still referring to FIG. 1A, an elongate releasing member 40 is disposed within the lumen 36 of the elongate sheath 30. The elongate releasing member 40 has a distal end 42 and a proximal end 44. The elongate releasing member 40 is formed from a flexible yet sturdy material that provides sufficient column strength to avoid breakage or kinking during the deployment process (described in more detail below). For example, the elongate releasing member 40 may be formed from one or more wires or filaments. The wire(s) may be formed from a metal or alloy such as NITINOL, titanium, platinum, stainless steel or the like. Alternatively, wire(s) may be formed from a polymer material such as polyimide, polypropylene, polyetheretherketone (PEEK), polytetrafluoroethylene (PTFE), and the like. FIG. 1A illustrates the elongate releasing member 40 as a single wire. It should be understood, however, the elongate releasing member 40 can be created as a coil or braided structure. The elongate releasing member 40 has a diameter that is less than the internal diameter of the elongate sheath 30. In one exemplary aspect, the elongate releasing member 40 is formed from NITINOL wire having an outer diameter on the order of around 0.002 inches.

As seen in FIG. 1A, the elongate releasing member 40 includes a tapered section 46 that reduces the effective diameter of the distal end 42 of the elongate releasing member 40. The reduction in the effective diameter of the distal end 42 may be accomplished by grinding down the distal end 42 of the elongate releasing member 40. Alternatively, a smaller segment of wire or the like may be bonded to a larger diameter proximal section. In one aspect, the distal end 42 of the elongate releasing member 40 may be formed from NITINOL having a diameter of around 0.002 inches.

As seen in FIG. 1A, the proximal end 34 of the elongate sheath 30 includes a locking member 90. The locking member 90 is used to fixedly secure the elongate releasing member 40 relative to the elongate sheath 30. This may be accomplished by the use of a cap or the like such as that illustrated in FIG. 1A that is bonded or otherwise engaged with the elongate releasing member 40. For example, the elongate sheath 30 may have a series of threads 38 on the exterior surface that engage with corresponding grooves 92 in the locking member 90. In the configuration shown in FIG. 1A, the elongate releasing member 40 cannot be moved in either the proximal or distal directions. In order to deploy the coil 14, the locking member 90 must first be released from the elongate sheath by unscrewing the same from the threads 38.

Still referring to FIG. 1A, the elongate sheath 30 may include an optional blocking member 50 disposed at a distal end 32 of the elongate sheath 30. The blocking member 50 may include an aperture 52 or passageway that is dimensioned to permit passage of a loop segment formed by a filament (discussed in more detail below) but not permit passage of the securing member 20 attached to the proximal end 18 of the coil 14. It also ensures proper engagement between the loop segment 70, the securing member 20, and the elongate releasing member 40. The blocking member 50 may be formed from a short segment of tubing such as, for instance stainless steel hypotube having an inner diameter of around 0.0045 inches and an outer diameter of around 0.0065 inches.

FIG. 1A illustrates a coil member 60 disposed in the distal end 32 of the elongate sheath 30. The coil member 60 may be formed from a short segment of coil having an outer diameter on the order of around 0.007 to around 0.010 inches. The coil member 60 may be formed from a metal or metal alloy including stainless steel, platinum, and NITINOL. In an alternative embodiment, such as that illustrated in FIG. 2A the coil member 60 may be exchanged for a tube member 76 (discussed in more detail below).

FIG. 1A illustrates a filament 64 having a first end 66, a second end 68, and a loop segment 70. As seen in FIG. 1A the two ends 66, 68 of the filament 64 are interposed between the exterior surface of the coil member 60 and the interior surface of the elongate sheath 30. In particular, the two ends 66, 68 of the filament 64 are fixedly secured to the coil member 60 and/or the elongate sheath 30. The two ends 66, 68 may be secured using a bonding agent 72 disposed between the coil member 60 and the elongate sheath 30. Depending on the materials used in the filament 64, the bonding agent 72 may include an adhesive, solder, or weld. The filament 64 can be formed from a polymer-based wire or cable such as, for instance polypropylene, polyetheretherketone (PEEK), polytetrafluoroethylene (PTFE), polyethylene terephthalate (PET), polyethylene naphthalene (PEN), NYLON and the like. The filament 64 may also be constructed of a metallic wire filament or cable such as stainless steel, NITINOL, titanium, platinum, and alloys. In one aspect, the filament 64 is formed from pre-shaped NITINOL wire having a diameter of 0.001 to 0.002 inches.

FIG. 1B illustrates an alternative device 10 in which the filament 64 does not pass through the aperture 52 of the blocking member 50. In this embodiment, the filament 64 is disposed between the exterior of the blocking member 50 and the interior surface of the elongate sheath 30. The filament 64 may be wedged in a frictional fit between the blocking member 50 and elongate sheath 30 to anchor the filament 64. A bonding agent 72 may be used to anchor the filament 64 relative to the elongate sheath 30. In this configuration, the blocking member 50 assists in securing the filament 64 to the elongate sheath 30.

In another embodiment, such as that illustrated in FIG. 2B, the coil member 60 may be omitted in its entirety. Namely, the filament 64 is anchored to the elongate sheath 30 by the use of a friction fit between the blocking member 50 and the interior surface of the elongate sheath 30. Alternatively, or in addition to, a bonding agent 72 may be applied to the region between the blocking member 50 and the elongate sheath 30 to fix the filament 64.

The loop segment 70 of the filament 64 is used in the delivery device 10 to secure the coil 14. The coil 14 is secured by passing the loop segment 70 through the securing member 20 attached to the proximal end 18 of the coil 14. The distal end 42 of the elongate releasing member 40 is then inserted into the portion of the loop segment 70 that passes through the securing member 20 (e.g., the distal most portion of the loop 70). The insertion of the distal end 42 may be accomplished by first pulling or pushing the loop segment 70 through the securing member 20 to ensure a small loop is formed for receiving the distal end 42. The elongate releasing member 40 is then advanced distally to pass through this small loop until the tip is inside the lumen or pocket 21 of the proximal end 18 of the coil 14. The coil 14 is then in a locked state relative to the elongate sheath 30. FIG. 1A illustrates the locked state between the coil 14 and the elongate sheath 30.

The “locked” coil 14 of FIG. 1A may be unlocked by proximally retracting the elongate releasing member 40. For example, a physician may pull on the elongate releasing member 40 using one hand while the other hand holds the elongate sheath 30. In some embodiments, a locking member 90 (e.g., as shown in FIGS. 1A, 2A, 4A, 4B, and 5) may be used to lock the elongate releasing member 40 relative to the elongate sheath 30 until deployment. The locking member 90 may be used to prevent premature detachment of the coil 14.

FIG. 2A illustrates an alternative embodiment of the delivery device 10. In this embodiment, the coil member 60 is replaced with a tube member 76. The tube member 76 may be formed using metallic hypotube, e.g., stainless steel or NITINOL hypotube. In one exemplary embodiment, the tube member 76 may be formed using a short segment (e.g., around 3 mm) of stainless steel hypotube having an internal diameter of around 0.0045 inches and an outer diameter of around 0.0065 inches. The tube member 76 may be disposed partially within the lumen 36 of the elongate sheath 30.

As seen in FIG. 2A, the first and second ends 66, 68 of the filament 64 is securely interposed between the exterior of the tube member 76 and the interior of the elongate sheath 30. Unlike the embodiment illustrated in FIG. 1A, the two ends 66, 68 of the filament 64 pass through the lumen 77 of the tube member 76 and are bent through approximately 180°. The first and second ends 66, 68 are secured using a bonding agent 72 between the exterior of the tube member 76 and the interior of the elongate sheath 30. The bonding agent 72 may include an adhesive, solder, or weld.

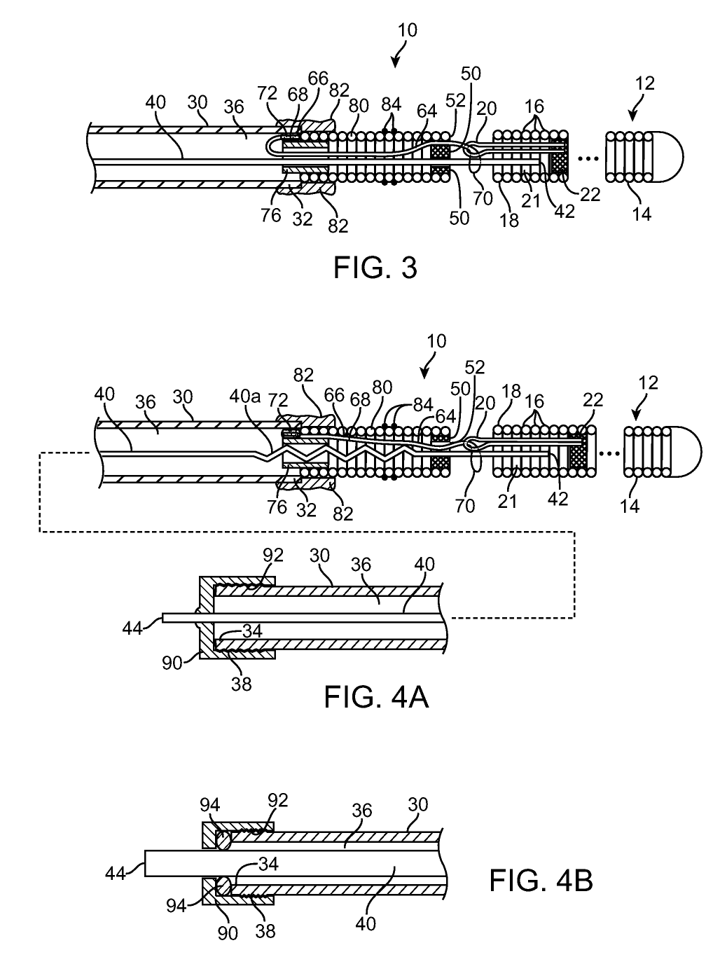

FIG. 3 illustrates yet another embodiment of a delivery device 10. In this embodiment, a tube member 76 partially disposed within the lumen 36 of the elongate sheath 30. As shown in FIG. 3, a portion of the tube member 76 projects distally with respect to the distal end 32 of the elongate sheath 30. A bonding agent 72 of the type described herein may be used to secure the tube member 76 to the interior of the elongate sheath 30. In addition, the same bonding agent 72 is used to fixedly secure the first and second ends 66, 68 of the filament 64. FIG. 3 illustrates an added coil segment 80 secured to the distal end 32 of the elongate sheath 30. The coil segment 80 may be secured to the exterior surface of the tube member 76 using a bonding agent (not shown) or may be simply press-fit over the outer surface. Alternatively, heat shrink tubing or the like (not shown) may be used for this joint. FIG. 3 illustrates a UV-curable adhesive 82 that coats the exterior of the elongate sheath 30, tube member 76, filament ends 66, 68, and the coil segment 80. The UV-curable adhesive 82 forms a solid bond amongst the coil segment 80, filament ends 66, 68, tube member 76, and the elongate sheath 30. The UV-curable adhesive 82 may include any number of biocompatible UV-curable adhesives.

Still referring to FIG. 3, the coil segment 80 may be formed from windings of a metallic coil (e.g., stainless steel, NITINOL, platinum, titanium, and the like). As one illustrative example, the coil segment 80 may be formed from 0.00175 inch stainless steel wire. The coil segment 80 may have an inner diameter within the range of around 0.004 inches to around 0.010 inches. The length of the coil segment 80 may vary from around 10 cm to around 30 cm. Of course, this range is exemplary and other dimensions falling outside the specific ranges above are also contemplated by the invention. The coil segment 80 imparts a degree of radial flexibility to the distal end of the delivery device 10. The delivery device 10 of FIGS. 3 and 4A also illustrates the use of a marker coil 84 on the coil segment 80. The marker coil 84, which is formed from a wire of radiopaque material such as platinum or platinum-based alloys, may take the form of windings of wire about the periphery or interior surface of the coil segment 80. As an alternative to the marker coil 84, a band, paint, or the like formed from a radiopaque material may also be used.

In the embodiment illustrated in FIG. 3, the blocking member 50 is secured to the distal end of the coil segment 80. The blocking member 50 may be disposed inside the interior of the coil segment 80 as is shown in FIG. 3. The blocking member 50 may be affixed using an adhesive, solder, weld, or the like as disclosed herein. The blocking member 50 also has one or more apertures 52 that permit the passage of the elongate releasing member 40 and/or filament 64 (e.g., loop segment 70). In this embodiment, the blocking member 50 is dimensioned to permit the passage of the filament 64 between the exterior of the blocking member 50 and the interior of the coil segment 80.

FIG. 4A illustrates another embodiment of a delivery device 10. In this embodiment, the elongate releasing member 40 includes a coiled, wavy-shaped, or zig-zag segment 40a. In this embodiment, prior to release of the coil 14, the elongate releasing member 40 is under compressive stress. The coiled, wavy-shaped, or zig-zag segment 40a lets compressive stress build within the elongate releasing member 40 without having the same buckle or fail. While FIG. 4A illustrates a relatively short segment 40a of the releasing member 40 that is coiled, wavy-shaped, or zigzagged, it should be understood that the segment may comprise a significant portion of overall elongate releasing member 40.

Still referring to FIG. 4A, in this embodiment, the distal end 42 of the elongate releasing member 40 passes into the lumen or pocket 21 of the proximal end 18 of the coil 14. As explained herein, by having the distal end 42 of the elongate releasing member 40 pass into the lumen or pocket 21 of the coil 14, this acts as a safety feature to prevent the distal end 42 from accidentally poking or puncturing the aneurysm 100.

The delivery device 10 of FIG. 4A also illustrates an aspect in which the first and second ends 66, 68 of the filament 64 are interposed between the tube member 76 and the elongate sheath 30 and coil segment 80. Unlike the embodiment illustrated in FIG. 3, the first and second ends 66, 68 are inserted directly between the tube member 76 and the elongate sheath 30/coil segment 80. Namely, there is no 180° bend or curve of the filament 64 like that illustrated in FIG. 3.

Still referring to FIG. 4A, the proximal end 34 of the elongate sheath 30 includes a locking member 90 of the type described herein. Alternatively, as illustrated in FIG. 4B, the locking member 90 may include a compressible O-ring 94 disposed between the proximal end 34 of the elongate sheath 30 and the locking member 90 or cap. As the locking member 90 is tightened down on the elongate sheath 30 (e.g., by screwing the locking member 90) the O-ring 94 is compressed thereby causing radial expansion or bulging of the O-ring 94 which frictionally engages with the elongate releasing member 40.

In an alternative embodiment, the locking member 90 is not directly bonded to the elongate releasing member 40. For example, the locking member 90 may be formed to include one or more pinching or grabbing elements that frictionally engage with the elongate releasing member 40 so as to prevent relative movement. For example, as the locking member 90 is screwed further on the threads 38, the pinching or grabbing elements may move toward one another until the elongate releasing member 40 is stopped from moving altogether. In still another alternative aspect, the locking member 90 may be temporarily affixed to the proximal end 34 of the elongate sheath 30. For example, a breakable or temporary bond between the locking member 90 and the sheath 30 may be released upon application of a threshold force (e.g., torsional or pulling force).

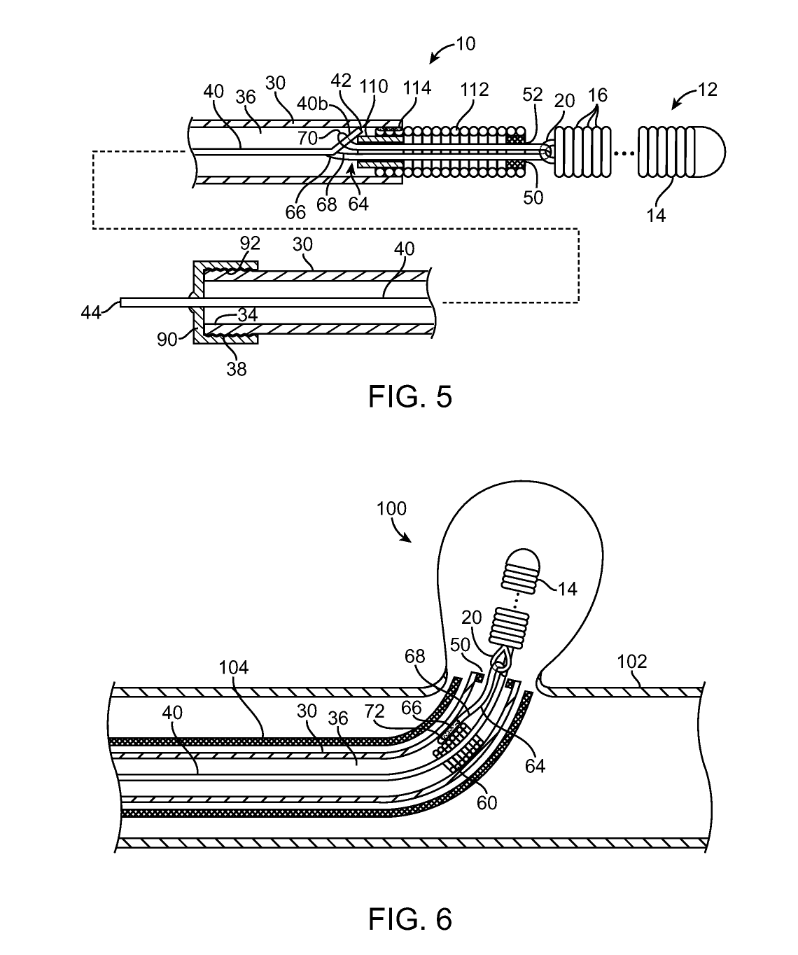

FIG. 5 illustrates yet another alternative embodiment of a delivery device 10. In this embodiment, a locking tube 110 is disposed in the distal end of the elongate sheath 30. The locking tube 110 may be made from a short segment of tubing (or even coil) that is secured to the distal end of the elongate sheath 30. For example, the locking tube 110 may be formed from a short segment of metallic hypotube (e.g., stainless steel or NITINOL). The locking tube 110 may be bonded directly to the elongate sheath 30 via an adhesive, solder, weld or the like. Alternatively, as shown in FIG. 5, the locking tube 110 may be bonded to a coil segment 112. The coil segment 112 is formed using a plurality of coil windings from a suitable material such as, for instance, stainless steel or NITINOL. The coil segment 112 imparts added flexibility to the distal end of the delivery device 10. The locking tube 110 may be affixed to the coil segment 112 using any number of techniques including, for example, an adhesive, glue, solder, or weld.

As illustrated in FIG. 5, at least a portion of the coil segment 112 is disposed in the lumen 36 of the elongate sheath 30. The coil segment 112 may be bonded to the interior surface of the elongate sheath with a bonding material 114 of the type disclosed herein (e.g., adhesive, solder, weld, or the like). A blocking member 50 is optionally included in the distal end of the coil segment 112 to prevent the proximal retraction of the coil 14 therethrough. The blocking member 50 includes an aperture 52 that is sized to permit passage of the filament 64 but not the coil 14.

In the embodiment of FIG. 5, the first and second ends 66, 68 of the filament 64 is secured directly to the elongate releasing member 40. The first and second ends 66, 68 of the filament 64 are bonded or otherwise affixed to the elongate releasing member 40 just proximal with respect to the distal end 42 of the of the elongate releasing member 40. In this regard, a short segment 40b of the elongate releasing member 40 projects distally from the bond or junction and is used to form a locking arrangement with the locking tube 110.

A locking configuration such as that illustrated in FIG. 5 is accomplished by passing the loop segment 70 of the filament 64 through the securing member 20 and backtracking in the proximal direction. The distal most section of the loop segment 70 is then placed around the short segment 40b of the elongate releasing member 40. The locking arrangement is formed because the short segment 40b of the elongate releasing member 40 is in contact with the exterior surface of the locking tube 110, thereby preventing the loop segment 70 from being withdrawn back through the locking tube 110.

In the embodiment of FIG. 5, the device 10 may be assembled by first affixing the locking tube 110 to the coil segment 112. The filament 64 that is secured to the end of the elongate releasing member 40 may then be loaded through the lumen of the locking tube 110 and coil segment 112 and passed through the securing member 20. The loop segment 70 of the filament 64 can then backtrack through the coil segment 112 and locking tube 110. The short segment 40b of the elongate releasing member 40 may then be inserted into the loop segment 70. The coil segment 112 (with attached locking tube 110) may then be secured directly to the elongate sheath 30 using a bonding material 114 as described herein.

In order to release the coil 14, the elongate releasing member 40 is retracted proximally so as to withdraw the short segment 40b of the elongate releasing member 40 from the loop segment 70. Prior to release, a locking member 90 of the type illustrated in FIG. 5 may need to be unscrewed or removed from the elongate sheath 30. By pulling the elongate releasing member 40 proximally, the loop segment 70 of the filament 64 is first separated form the short segment 40b of the elongate releasing member 40 and then withdrawn from the securing member 20. Once the loop segment 70 is free from securing member 20, the coil 14 is free from the device.

FIG. 6, illustrates a process of delivering the coil 14 to an aneurysm 100 in a blood vessel 102. As seen in FIG. 6, a catheter 104 device such as a microcatheter is positioned within the vessel 102 so as to place the coil 14 within the aneurysm 100. The device 10 of the type described herein is then advanced through the catheter 104. The device 10 may be pre-loaded in an introducer sheath or the like (not shown). The device 10 is then advanced to place the coil 14 in the aneurysm 100. One or more radiopaque markers (e.g., coils 84) located on the delivery device 10 and/or catheter 104 may be used to aid the physician in positioning the device 10 for deployment of the coil 14. FIG. 6 illustrates the coil 14 in a locked arrangement with the elongate releasing member 40. The coil 14 is being held on the distal end of the elongate sheath 30 by the filament 64 that is releaseably secured to the elongate releasing member 40. The coil 14 is then released into the aneurysm 100 by proximal retraction of the elongate releasing member 40.

One advantage of the delivery system 10 described herein is that a pull-to-release process is used to deploy the coil 14. Because a pulling motion is used, there is no risk of poking or puncturing the aneurysm 100 that is inherent in push-based delivery devices. In addition, because the coupling between the coil 14 and the elongate releasing member 40 is mechanical, detachment is faster than electrolytic-based delivery devices. Moreover, in certain embodiments the distal end 42 of the elongate releasing member 40 is protected within the interior of the coil 14. In addition, by using a coil segment 80 on the distal end of the device a semi-articulating main junction is created, thereby reducing the chance of microcatheter kick-back and avoiding coil-catching micro-catheter when coil retrieval is necessary. Finally, the nature of coupling between the coil 14 and the elongate releasing member 40 produces an atraumatic, smooth release of the coil 14 during deployment.

While embodiments of the present invention have been shown and described, various modifications may be made without departing from the scope of the present invention. The invention, therefore, should not be limited, except to the following claims, and their equivalents.

What is claimed is:

1. A device for delivering an occlusive element comprising:
an occlusive element including a securing member positioned at a proximal end thereof;
an elongate sheath having a distal end and a proximal end and lumen extending between the distal and proximal ends;
an elongate releasing member disposed within the lumen of the elongate sheath, the elongate releasing member including a proximal end and a distal end, the elongate releasing member being moveable within the lumen of the elongate sheath;
a filament having first and second ends secured to the elongate sheath so as to form a loop segment; and
wherein the device includes a locked state and an unlocked state, the locked state formed when the loop segment passes through the securing member and the distal end of the elongate releasing member engages with the loop segment passing through the securing member and wherein the unlocked state is formed when the distal end of the elongate releasing member is retracted proximally from the loop segment.

2. The device of claim 1, further comprising a coil member disposed in the lumen of the elongate sheath at the distal end, the first and second filament ends being interposed between the exterior of the coil member and the interior of the elongate sheath.

3. The device of claim 1, further comprising a tube member disposed in the lumen of the elongate sheath at the distal end, the first and second filament ends being interposed between the exterior of the tube member and the interior of the elongate sheath.

4. The device of claim 1, wherein the filament is one of a polymer or metallic material.

5. The device of claim 4, wherein the filament comprises one of stainless steel, NITINOL, titanium, platinum, or an alloy of the same.

6. The device of claim 1, further comprising a coil segment bonded to the distal end of the elongate sheath.

7. The device of claim 1, wherein the elongate releasing member comprises a wire.

8. The device of claim 1, wherein the elongate releasing member comprises a narrowed distal tip portion.

9. The device of claim 1, further comprising a blocking member disposed in a distal end of the elongate sheath, the blocking member including an aperture sized to permit passage of the elongate releasing member and filament but not the occlusive element.

10. The device of claim 6, further comprising a blocking member disposed in a distal end of the coil segment, the blocking member including an aperture sized to permit passage of the elongate releasing member and filament but not the occlusive element.

11. A device for delivering an occlusive element comprising:
an occlusive element including a securing member positioned at a proximal end thereof;
an elongate sheath having a distal end and a proximal end and lumen extending between the distal and proximal ends;
an elongate releasing member disposed within the lumen of the elongate sheath, the elongate releasing member including a proximal end and a distal tip, the elongate releasing member being moveable within the lumen of the elongate sheath;
a blocking member disposed in a distal end of the elongate sheath;
a filament having first and second ends fixedly interposed between the blocking member and the elongate sheath so as to form a loop segment; and
wherein the device includes a locked state and an unlocked state, the locked state formed when the loop segment passes through the securing member and the distal end of the elongate releasing member engages with the loop segment passing through the securing member and wherein the unlocked state is formed when the distal end of the elongate releasing member is retracted proximally from the loop segment.

12. The device of claim 11, wherein the filament is one of a polymer or metallic material.

13. The device of claim 12, wherein the filament comprises one of stainless steel, NITINOL, titanium, platinum, or an alloy of the same.

14. The device of claim 11, wherein the blocking member comprises a tube.

15. The device of claim 14, wherein the blocking member includes an aperture sized to permit passage of the filament but not the occlusive element.

16. The device of claim 11, wherein the elongate releasing member comprises a wire.

17. The device of claim 11, wherein the elongate releasing member comprises a narrowed distal tip portion.

18. A method of loading a vaso-occlusive coil on a delivery device comprising:
providing a vaso-occlusive coil having a securing member disposed at a proximal end thereof;
providing an elongate sheath having a distal end and a proximal end and lumen extending between the distal and proximal ends;
providing a filament having first and second ends secured to a distal portion of the elongate sheath to form a loop segment;
providing an elongate releasing member within the lumen of the elongate sheath, the elongate releasing member being moveable within the lumen of the elongate sheath;
introducing the loop segment of the filament through the securing member of the vaso-occlusive coil; and
inserting a distal end of the elongate releasing member through the portion of loop segment inserted through the securing member so as to affix the vaso-occlusive coil relative to the elongate sheath.

19. The method of claim 18, further comprising the step of loading the elongate sheath in an introducer.

20. The method of claim 19, further comprising the step of advancing the introducer within a catheter disposed in a body so as to position the vaso-occlusive coil within an aneurysm.

21. The method of claim 18, further comprising the step of proximally retracting the elongate releasing member so as to release the vaso-occlusive coil within the aneurysm.
